(19) United States
(12) Patent Application Publication
(10) Pub. No.: US 2004/0106101 A1
(43) Pub. Date: Jun. 3, 2004

(54) System and method for quality control of a shipped neural cell culture on a microelectrode array

(76) Inventor: Daron G. Evans, Dallas, TX (US)

Correspondence Address: CHALKER FLORES, LLP, 12700 PARK CENTRAL, STE. 455, DALLAS, TX, 75251, US

(21) Appl. No.: 10/370,660

(22) Filed: Feb. 20, 2003

Related U.S. Application Data
(60) Provisional application No. 60/430,409, filed on Dec. 2, 2002.

(51) Int. Cl. G06F 17/60; C12Q 1/70; G06F 19/00; G01N 33/48; G01N 33/50

(52) U.S. Cl. 435/5; 705/2; 702/19; 800/20

(57) ABSTRACT

The present invention provides a system and method for providing and using quality control information pertaining to a cell culture. The system includes a chamber (106) containing the cell culture (104) and a unique identifier (116) disposed on the chamber (106) corresponding to a data file (114) containing the quality control information pertaining to the cell culture before an event. One method includes obtaining quality control information from the microelectrode array before the event (132), storing the quality control information in a data file (134), associating a unique identifier with the microelectrode array that corresponds to the data file (136), and providing access to the data file via the unique identifier after the event (138). Another method includes obtaining quality control information from the microelectrode array after the event (162), accessing the quality control information obtained before the event (164), and comparing the quality control information obtained before the event with the quality control information obtained after the event (166).

### REFERENCE TO A COMPUTER PROGRAM LISTING

This application claims priority to U.S. Provisional Patent Application Serial No. 60/430,409, filed Dec. 2, 2002. A computer program listing appendix “A” on a compact disc is included and the material of the disc is incorporated herein by reference. A total of two (2) compact discs are submitted, one original and one duplicate identified as “Copy 1” and “Copy 2”, each disc containing three (3) data files identified as “PLAC.txt” (2 KB created Feb. 18, 2003), “FEC.txt” (5 KB created Feb. 18, 2003) and “Sort.txt” (18 KB created Feb. 18, 2003).

### TECHNICAL FIELD OF THE INVENTION

The present invention relates in general to the field of quality control during the shipment of a cell culture, and more particularly, to the shipment of cell cultures that are used in neuroactivity detection techniques for the detailed analysis of neuronal signal transduction pathways and for large-scale reproducible analysis.

### BACKGROUND OF THE INVENTION

Without limiting the scope of the invention, the background of the invention is described in connection with the recording and analysis of neuronal action potentials using substrate integrated, thin film electrodes, as an example.

The first recordings of neuronal action potentials using substrate integrated, thin film electrodes were made as early as 1977 (Gross, et al. 977). Subsequent research has led to multi-channel investigations of network dynamics and their applications. Indium-tin oxide was introduced later as a viable microelectrode material and was designed and tested for recording in life support chambers (Gross and Schwalm, 1995). These networks were used to explore stimulation of networks through the recording electrodes (Gross et al., 1994).

Linked dual, age-matched neuronal networks have been grown on microelectrode arrays with for possible uses as biosensors (Gross et al., 1995). A practical and realistic use of neural networks is in their application as physiological function deficit detectors. Due to electrophysiological mechanisms, neurons represent efficient transducers for detecting and recording the dynamics of cell death, receptor-ligand interactions, alterations in metabolism, cell signal transduction cascade events, and generic membrane perforation processes. As such, mammalian networks in culture, devoid of extra-neuronal homeostatic protection mechanisms, function as reliable and highly sensitive detectors of any toxicant capable of interfering with autonomic life support, neuromuscular functions, and even behavior.

Although single neurons are often vulnerable and unreliable, networks of neurons may be used to form robust, fault-tolerant, spontaneously active dynamic systems with high sensitivity to their chemical environment. Networks in culture generate response profiles that are concentration and substance specific and react to a broad range of compounds. Pharmacologically and toxicologically, neuronal networks are representative of the parent tissue.

Portable cell-based biosensors (Gray et al., 2001) have been developed to provide toxicity monitoring. Neuronal cells can be used for these portable biosensors. A shipping chamber is used to supply these biosensors with cell. The cell cultures shipped on electrode plates require 30 minutes to 1 hour of time to select the active electrodes and to identify the action potential single patterns within the active electrode. This step must be performed in order to begin experimentation. As a result, there is a need for a system and method for providing and using quality control information pertaining to cell cultures so that the receiving company may eliminate this set up time and or may not use a skilled neuroscientist to perform the experiment. Quality control procedures would enable users to monitor the effects of shipping on the cell cultures used for the portable cell-based biosensor, and for fixed test stations using neuronal cells grown on microelectrode arrays.

### SUMMARY OF THE INVENTION

The present invention provides a method to assess the quality, based on the recorded electrical activity, of cell cultures grown on microelectrode arrays to be tracked over time to determine the effect of events on cell culture in a chamber. The events could be the shipment of the cell culture in a shipping chamber, the addition of a compound to the cell culture medium, or some other experimental or commercial procedure performed on an electrically active cell culture grown on a microelectrode array. The testing may involve an automated pre- and post-shipment subroutine to evaluate the spontaneous or stimulated activity of a cell culture, which could be a neuronal cell culture grown on a microelectrode array. The cell culture grown on the microelectrode array is communicably coupled to a data capture unit. A processor is communicably coupled to the data capture unit and one or more input/output devices. The microelectrode array, which can be a MEA detector, is capable of supporting wild-type or genetically modified neuronal cells and measuring neuronal activity. The microelectrode array can also be a chamber having a fluid input connected to a perfusion system or having fluid in a closed chamber. The processor, which can be a computer, measures the neuronal activity of the neuronal cells.

The software subroutine records data from the neuronal cells on the microelectrode array. The subroutine characterizes the quality of the cell culture, based on extracellular voltage recordings, before and after an event, which could be the shipment of the cell culture in a shipping chamber. The pre- and post-event characterizations allow users to track the changes in the electrical activity patterns of the cell culture that were induced by an event, such as the shipment of the cell culture.

In addition, the present invention provides a method of linking the pre- and post-event data file to the cell culture. The unique identifier or label corresponds to a data file that will record the quality characterization. The unique identifier or label can be a bar code, a magnetic identifier, or some other system that enables a extracellular recording test station to automatically identify the cell culture and match it with the appropriate data file, which could be on magnetic medium or stored in an internet-accessible location. The unique identifier or label of cell culture chamber, which could be a shipping chamber, is identified by the extracellular recording test station to enable the test station software to automatically retrieve the linked data file and incorporate the data into the quality characterization.

The present invention also provides a system for providing quality control infornmation pertaining to a cell culture that includes a chamber containing the cell culture and a unique identifier disposed on the chamber corresponding to a data file containing the quality control information pertaining to the cell culture before an event. The quality control information may include baseline testing information, hardware and software settings or other relevant information. For example, the quality control information may include an identification of each active electrode on which the cell culture are present and one or more waveform templates for each active electrode. The unique identifier can be a memory device containing the data file, such as a flash memory or a USB key. The unique identifier may also correspond to a computer readable medium where the data file is stored, such as a floppy disk, a smart card or a CD-ROM, to name a few. Moreover, the unique identifier can be a bar code, a series of alphanumeric characters or other identification mechanism. The data file may be accessible via the Internet or be delivered via e-mail. The chamber can be a shipping chamber, a testing chamber or serve as both. The cell culture can be neuronal cells, such as an embryonic stem cell from a knock-out, knock-in, over-expressing transgenic, under-expressing-transgenic, a conditional knockout, a mutant and the like, or be from an animal knock-out, knock-in, over-expressing transgenic, under-expressing-transgenic, a conditional knockout, a mutant and the like. The neuronal cells may be selected from the frontal cortex, the auditory cortex, the visual cortex, the hippocampus or the spinal cord.

In addition, the present invention provides a method for providing quality control information obtained from a microelectrode array before an event by obtaining quality control information from the microelectrode array before the event, storing the quality control information in a data file, associating a unique identifier with the microelectrode array that corresponds to the data file, and providing access to the data file via the unique identifier after the event. This method may also include growing a cell culture on the microelectrode array and assembling a chamber that includes the cell culture grown on the microelectrode array and that can be used to ship and test the cell culture.

Moreover, the present invention provides a method for using quality control information obtained from a microelectrode array before an event by obtaining quality control information from the microelectrode array after the event, accessing the quality control information obtained before the event and comparing the quality control information obtained before the event with the quality control information obtained after the event.

The present invention also provides a method for using quality control information obtained from a microelectrode array by obtaining quality control information from the microelectrode array before an event, storing the quality control information obtained before the event in a data file, associating a unique identifier with the microelectrode array that corresponds to the data file, obtaining quality control information from the microelectrode array after the event, accessing the quality control information obtained before the event via the unique identifier, and comparing the quality control information obtained before the event with the quality control information obtained after the event.

In addition, the present invention provides a method for providing an automated quality characterization of a cell culture grown on a microelectrode array for the purpose of measuring the effects on the cell culture before and after an event. The method includes the steps of growing the cell culture from a wild-type or genetically modified animal on the microelectrode array, assembling a chamber that includes the cell culture grown on the microelectrode array and that can be used to ship and test the cell culture, measuring an electrical activity of the cell culture before an event in order to characterize a quality of the cell culture, storing quality control information pertaining to the cell culture, which includes both software settings and the measured electrical activity of the cell culture, on a computer readable medium for the purposes of reference at the time of subsequent testing of the cell culture, measuring the electrical activity of the cell culture after the event, and comparing the quality characterization measurements from the cell culture before and after the event to assess the effects of the event on the cell culture and the usability of the cell culture for future experiments.

### BRIEF DESCRIPTION OF THE DRAWINGS

For a more complete understanding of the features and advantages of the present invention, reference is now made to the detailed description of the invention along with the accompanying figures in which corresponding numerals in the different figures refer to corresponding parts and in which:

FIG. 1A is a block diagram of a system in accordance with the present invention;

FIG. 1B is a flow chart illustrating various methods in accordance with the present invention;

FIGS. 2A, 2B, 3A and 3B illustrate typical microelectrode arrays that can be used in connection with the present invention;

FIG. 4 is a photograph of neuronal cells grown on a microelectrode array in connection with the present invention;

FIG. 5 is a photograph of a standard shipping chamber in accordance with the present invention;

FIGS. 6A and 6B are cross sectional drawings of a shipping chamber in accordance with the present invention in an open chamber configuration and closed chamber configuration, respectively;

FIG. 7 illustrates the integration of a shipping chamber with a data capture unit in accordance with the present invention;

FIG. 8 is a cross sectional drawing of a shipping chamber (open chamber configuration) connected to a data capture unit in accordance with the present invention;

FIG. 9 illustrates a screen from a typical extracellular recording software program in accordance with the present invention;

FIG. 10 is a flow chart illustrating the pre-shipment testing method to characterize the neuronal cell culture in accordance with the present invention;

FIG. 11 is a flow chart outlining the post-shipment testing method in accordance with the present invention;

FIG. 12 illustrates a pre- and post-event channel data comparison in accordance with the present invention; and

FIG. 13 illustrates a pre- and post-event unit waveform comparison in accordance with the present invention.

### DETAILED DESCRIPTION OF THE INVENTION

While the production and application of various embodiments of the present invention are discussed in detail below, it should be appreciated that the present invention provides many applicable inventive concepts that may be embodied in a wide variety of specific contexts. The specific embodiments discussed herein are merely illustrative of specific ways to make and use the invention and do not delimit the scope of the invention.

The present invention takes advantage of mammalian neuronal networks grown on substrate integrated microelectrode arrays (MEAs). Primary cultures from dissociated tissue have superior adhesion to the recording substrate, stability during recording, longevity, and number of active channels that can be observed on the spike level with good signal-to-noise ratios. These observations include (1) neuronal networks most likely respond to ANY substance that has a major effect on central nervous system functions, (2) the sensitivities and efficacies are comparable to those causing responses in vivo, (3) false positives and false negatives appear minimal and, in many cases, may be predictable, (4) agent response profiles are reproducible and, with improved data processing, may identify mechanisms and classify an increasing number of substances, and (5) a simple, reliable warning system can be constructed.

Neuronal Network Biosensors (NNBS) are living nerve cell networks growing on arrays of substrate integrated miroelectrodes in cell culture. The networks are constantly spontaneously active and allow long-term (months) monitoring of action potential (AP or “spike” ) patterns from as many as 64 channels simultaneously. These networks, as isolated neural tissue, devoid of the blood-brain barrier and other non-neuronal homeostatic mechanisms are highly sensitive to their environment and respond to chemical and physical changes in the life support medium with increases, decreases, or pattern changes in their spike activity. In addition, AP amplitude decreases reflect metabolic changes that lead to a reduction of the membrane potential.

The readout from such systems is any change from the normal activity that a particular culture has established. Not all networks have identical starting (or native) activity as long as they are spontaneously active. Note that the NNBS does not have to generate exactly the same patterns as the tissue in vivo. It is only necessary to establish a “cultured network correlate response” that can be reliably elicited from networks in response to a certain class of compounds for which the physiological effect is known. For high-throughput application, large numbers of integrated microculture chambers containing a variety of neural and non-neural tissues with a microfluidic system that can mimic normal physiological routing and interactions may be developed.

The NNBS is a generic sensor that mimics pharmacologically the nervous system of an animal. For example disinhibitory compounds all enhance bursting and regularization of the burst pattern. Such compounds all cause epilepsy in mammals. Therefore regularization of burst patterns in cultures and epilepsy may be correlated.

Microelectrode arrays (MEAs) come in single and dual network designs. The dual networks provide a control culture that can monitor the life support system or provide a second network. Use of a dual network array allows the growth of “twin networks” that have the same seeding date, seeding pool, and feeding manipulations. Cultures grown on the dual network array grow under the same medium in isolated adhesion areas and are separated into separate medium pools only upon assembly of the chamber. A dual network design may use a 5×5 cm plate and edge contact arrangement. Each network is served by, e.g., 32 microelectrodes.

Burst pattern changes in response to an agent may be recorded as integrated spike data displayed on a chart recorder. The results from different studies may be recorded and catalogued such that the molecular signature of such agent(s) may be used in sampling unknowns. Examples of compounds that may be tested and catalogued include, e.g mind-altering drugs such as the cannabinoids or even substances that have subtle effects generally detected as tinnitus, hallucinations, vertigo, irritability, loss of concentration, and minor loss of muscle coordination.

Generally, networks with 1,000 to 5,000 neurons growing adhesion areas with 3 to 4 mm diameters may be used. These systems can lose a significant percentage of neurons without showing any deficit in their spontaneous activity or their pharmacological responses.

In operation, neuronal cells over the recording matrix (1 mm² area) and axons from outside the recording matrix supply the spontaneous activity. Despite density fluctuations, a stabilization of neuronal counts past 30 days is obtained. Neuronal losses are approximately 20% in 100 days (6% per month). Neuronal counts include the total number of active signals recorded from the culture. The exclusion of a signal from the count does not signify neuronal cell death, only a loss of activity. NNBS responses are generally histiotypic, that is, the networks act as physiological sensors that can predict the effects of unknown compounds on the nervous system and allow an extrapolation to behavioral deficits.

Furthermore, because the networks express the same receptors and channels found in the parent tissue they have been found to respond very much like the nervous system of an animal would respond. Networks growing in culture on substrate integrated microelectrode arrays serve to link the molecular biochemistry of the network with results from whole animal physiology. The networks of the present invention may be used to provide rapid and accurate information on one or more pharmacological or toxicological changes.

Although a typical network has between 1,000 and 5,000 neurons, the number of inputs in, e.g., a 64-amplifier recording system limit analysis to 64 sites of the network. Using spike separation, e.g., it is possible to record from more than 100 individual neurons, as many electrodes carry signals from more than one axon. With the present 32 DSPs (digital signal processors), 32 channels may be selected for digitizing. Under optimal separation conditions, a user may record a maximum of 128 active units (4×32). For most sensing uses, however, such a high number of channels is more than sufficient.

Responses to toxicants are usually global, i.e., all channels are affected in a highly similar manner. Such responses can be detected reliably (and be quantified) with data from 10 to 20 channels. Responses to hallucinogens may be more complex by generating unit-specific responses where groups of different neurons respond differently. Therefore, the number of electrodes required to give a statistically sound representation of the network depends on the complexity of the response. Fortunately, in toxicology the end points of many, if not most, responses are relatively simple.

Response Quantification. Response quantification occurs generally in three stages: (1) detection, (2) classification and (3) identification. Detection will depend on independent multivariate z-scores, i.e. on changes of any activity variable or group of activity variables that exceed 2 or 3 standard deviations of the reference activity. Classification is based on simple, but major physiological responses that will be identified as inhibitory, disinhibitory, and excitatory. Whereas inhibition and excitation depend heavily on spike rate, disinhibition (which emerges during generation of epileptiform activity) requires measurement of pattern regularity. An interesting distinction between excitation and disinhibition is that both types of responses increase spike production, however, the resulting patterns are radically different. Excitation increases activity without favoring regularity. Disinhibition (substances that silence inhibitory circuits by blocking GABA and or glycine receptors) always generates bursting and high burst pattern regularity.

Identification after classification is a complex task and requires extensive scrutiny of response profiles and application of a variety of methods that have not yet been completely identified. Response profile matching with those generated by known compounds is certainly an essential step. Using the present invention, a number of systems may be tested and quantified for detection, classified and identified. Often, a single unique feature of the profile may identify a compound, e.g, botulinum toxin A. The features of a Toxin A response includes a long, concentration-independent delay and slow, but irreversible decline of all activity that is highly unique. The delay is caused primarily by receptor dependent internalization of this large protein proenzyme.

Biostatistics. A Plexon MNAP 64 channel workstation using Plexon data acquisition software and the NEX Technologies Neuroexplorer program may be used for data acquisition and analysis. The Plexon system allows action potential (AP or spike) discrimination with 32 digital signal processors that simplify the data before it reaches the host computer. In optimal cases, four different active units could be distinguished per channel resulting in a maximum capacity of 128 logical channels available for analysis.

Normally, the 64 electrode MEA yields an average of 30 channels with good signal-to-noise ratios where at least one or two units can be clearly identified and separated on each channel. The 64 electrode MEA yields an operational maximum of 60 logical channels. Both spike time stamps and waveforms may be collected for analyses of pattern changes and influences on membrane potentials or voltage-gated channel performance that would alter the AP wave shape. Data can be exported to Excel, Kaleidagraph, and Matlab (among many other programs for plotting or further statistical analyses).

The multichannel enviromnent is still somewhat unique in electrophysiology and effective methods for optimal network analyses are evolving. The following basic montage of plots for characterization of the network dynamics may be used: (1) temporal evolution of burst and spike rates in terms of cross channel means and their standard deviations; (2) dose-response curves based on spike production on all channels; (3) temporal evolution of burst variables (a) duration, (b) period, (c) max spike frequencies in bursts, and (d) burst coordination across channels. Because studies can last anywhere from 15 minutes to more than 48 hours and network responses need to be followed in real-time, it is convenient to form “minute means (MM)” for all burst variables (except rate, which is a scalar) and follow the network responses in terms of one minute steps. These minute means are grouped into “experimental episode means (EEM)” that are then compared to the reference activity mean.

The system is often adjusted for substance-specific effects that can influence the final analysis. Often it is necessary to select a “response stationarity” for best results. For example, synaptic receptor-mediated responses are generally rapid, but often decay as the network adapts or as the substance is degraded enzymatically. Conversely, metabotropic receptor-mediated effects are generally slower in changing network activity, but will reach a maximum effect for a variable period of time. In addition, response times are concentration-dependent. Therefore, in this environment, a fixed time protocol must be supplemented by selecting periods of network stationarity, where activity establishes a constant pattern. Therefore “experimental episode means” may be calculated from time periods that are shorter than the episode defined by test substance application to the next medium change.

Networks Statistics. The classical spike train statistics of NEX may be supplemented with more useful network statistics. For example, by using minute means that lead to test episode means, and subsequently cross channel (or network) episode means, and the use of coefficients of variation.

Chip Design. MEAs may be fabricated using, e.g., chromium masks and may be obtained from Photronix, Colorado Springs, Colo. Further customization may be useful for specific applications. MEAs are made often from a rugged glass carrier plate, indium-tin oxide conductors with gold deposits at exposed sites and dimethyl polysiloxane as insulator. MEAs have been found to have a lifetime of several years and are not toxic. MEAs are remarkably rugged, some have been used for 8-10 cycles of use, e.g., 2 months under warm medium for each cycle, followed by autoclaving and flaming to activate the surface before decoration with polylysine and laminin, without an appreciable loss of function.

Sample Collection and Preparation. A generic sensor may be designed and used that has the capability to sample water, air (with appropriate concentration and elution steps), and even human serum and urine. The NNBS is combined with a sample and a 2× concentrations of supply medium in order to obtain a maximum concentration of a potential toxicant. It may even be feasible to obtain a 25% medium, 75% sample water ratio or even higher concentrations of media depending on the solubility of the basic components of the media and their interaction with the sample.

Flow Rates. Closed chambers often operate at 20 to 40 μl per min. This flow rate is dictated by the small laminar flow chamber design that has only a 300 μm space between the cells and the glass window. Higher rates cause shear stress of cells, channel destabilization and changes in activity. Over long periods of time the shear stress will promote Ca⁺⁺ entry and cell death. As these flow rates are too slow for rapid sample detection, the chambers may be modified to accommodate a flow rate of 1 ml per min. If tubing distances are kept to a minimum (such as 20 cm between sample stores and network and small inner diameter tubing is used (1 mm), then a flow rate of 1 ml/min translates to a sampling time of approximately 38 sec.

In operation, the following conditions may be used in a chamber for use with the present invention, namely:

| | |
|---|---|
| Medium Supply: | 200 ml (2X concentration) |
| Internal Water Supply: | 200 ml |
| Total Medium Supply: | 400 ml |

(A) Flow rate through recording chamber at 20-40 μl/min (2.4 ml/hr)

| | |
|---|---|
| Total Running Time with medium voided: | 181 hrs (7.5 days) |

Total Running Time (at 40 μl/min) with medium recirculation at a medium usage (voided) of 10 ml/week: 40 weeks (10 months)

(B) Flow rate of 1 ml/min (in modified chambers)

| | |
|---|---|
| Total Running Time with medium voided: | 400 min |

Total Running Time with medium recirculation (10 ml per week used & voided): 40 weeks

The above conditions may or may not take sampling into consideration. For example, samples with potential toxic substances are best avoided prior to sampling. Test samples, however, often need to be circulated for a minimum of about 30-360 min. These parameters may be varied depending on the detection time required for pattern stabilization, classification, and possibly identification.

Constant Bath. It is also possible to perform testing with a constant bath chamber.

Medium is placed in the chamber (1 ml or 2 ml, depending on the chamber design). Compound aliquots are added in quantities less than 10 μl, giving whole bath compound concentrations in the pico- to micro-range.

Chambers have been created to ship the cultures from a cell culture laboratory to a testing facility. Cell cultures have been shipped from Dallas to San Diego, Dallas to Washington, D.C., from Dallas to Germany. The number of active channels (which correspond to individual neurons) from which action potentials can be recorded, changes after the cell culture is stressed. Shipping the cell culture causes stress to the cell culture.

During the shipping process, the cell cultures receive acceleration shocks, undergo temperature changes, undergo pressure changes and undergo pH changes, which can be a function of CO₂ partial pressure in the shipping container, depending on the cell culture medium used while shipping.

In order to perform neuroactivity testing, the cell culture needs to meet certain criteria. Criteria for usable neuronal cultures have been outlined by the Center for Network Neuroscience, as listed in Table 1. Neuronal cultures that do not meet these standards may not produce reliable neuroactivity data and can not be used for experimentation. It is therefore important to be able to measure the quality of a neuronal cell culture use for extracellular recording, especially after an event that stresses the cell cultures. Cell cultures that do not pass testing hurdles may be recovered through time and medium changes.

TABLE 1
Quality control parameters

| Quality Control Parameter | CNNS Criteria |
|---|---|
| Neuronal network visual appearance | Smooth cell bodies |
| | Visible nuclei |
| | No fungal contamination |
| Neuronal network electrode coverage | Greater 90% coverage of cell on |
| | electrodes |
| Cell density | Greater than 200 cells per sq. mm. |
| Signal-to-noise ratio | 3:1 |
| | 15 or more channels |
| Temporal stability | Less than 20% change in spike |
| | rate, averaged across the entire |
| | network |
| Hydrodynamic stability | Less than 20% change in spike |
| | rate, averaged across the entire |
| | network, in response to a 0.05 |
| | ml/sec flow rate |
| Osmotic stability | Less than 20% change in spike |
| | rate, averaged across the entire |
| | network, in response to a 2% |
| | dilution of the medium |

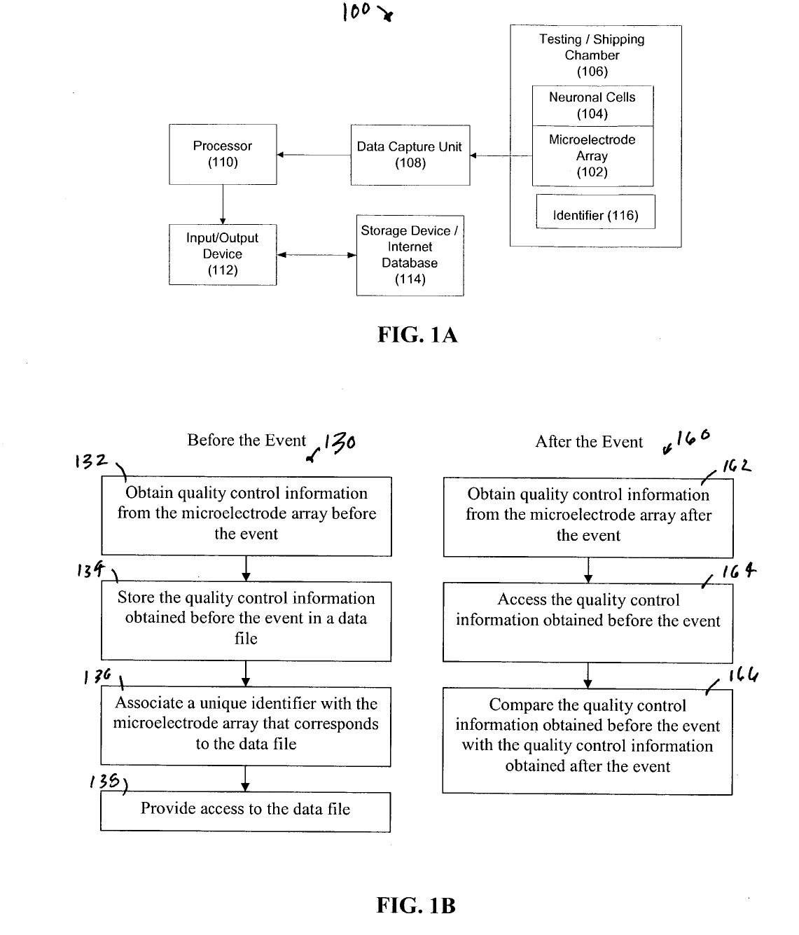

Now referring to FIG. 1A, a block diagram of a system 100 in accordance with the present invention is shown. The system 100 for testing the neuronal effects of a compound includes a microelectrode array 102, a data capture unit 108 communicably coupled to the microelectrode array 102, a processor 1 10 communicably coupled to the data capture unit 106 and one or more input/output devices 112 communicably coupled to the processor 110. The input/output device stores data either on electronic medium or on an inten-et accessible database 114. The microelectrode array 102, which can be a MEA detector, is capable of supporting wild-type and genetically modified neuronal cells 104 and measuring neuronal activity. The microelectrode array 102 can also be a chamber having a fluid input connected to a perfusion system. The processor, which can be a computer, compares the neuronal activity of the neuronal cells 104 in the presence and absence of a compound. The testing/shipping chamber 106 includes both the microelectrode array 102 and the neuronal cells 104. The shipping/testing chamber 106 can be used as a testing chamber in multiple types of systems with various data capture units 108. An identifier 116 is placed on the testing/shipping chamber 106 that either contains or refers to baseline testing information obtained from the microelectrode array 102 before shipping.

The present invention eliminates the time consuming and labor intensive step of electrode and wave form identification for cell cultures on a microelectrode array. The shipper of the cell cultures pre-identifies the active electrodes and their corresponding action potential signals. This present invention also allow the recipient of the cell cultures to plug-and-play the cell culture into the recording device so that they can: quickly use shipped cultures; reduce the skill require to perform extracellular recordings; and add more automation to the extracellular recording process.

The present invention provides pre-selection of active electrodes and wave forms from an electrically active (producing action potentials) cell culture and storing the information to be sent with a shipped cell culture. The cell culture recipient can then use the data to facilitate the use of the cell culture for experimentation. This data can be stored in a memory device on or within the shipping chamber, such as flash memory, a USB key or other plug-in memory device. The data may also be stored on a computer readable medium, such as a floppy disk, smart card or CD-ROM, that is shipped with the shipping chamber. In addition, the data may be sent to the recipient via e-mail or made accessable to the recipient via Internet downloads based on an identifier, such as a bar code or series of alphanumeric characters imprinted on the shipping chamber or label, or a physical deformation on the shipping chamber. The data file would include the active electrodes as well as one or more wave form templates for each active electrode. The test station can automatically obtain the data from the data file. The software at the receiving end would use these as a starting point from which to begin forming its neuroactivity baseline for experimentation. The data may also be used by extracellular recording software to perform automatic spike identification.

Now referring to FIG. 1B, a flow chart illustrates various methods of the present invention. Specifically, a method for providing quality control information before an event is shown in flow chart 130, and a method for using quality control information after an event is shown in flow chart 160. These two methods can be combined for form a single method that is used before and after an event. The method for providing quality control information obtained from a microelectrode array before an event 130 includes the steps of obtaining quality control information from the microelectrode array before the event in block 132, storing the quality control information in a data file in block 134, associating a unique identifier with the microelectrode array that corresponds to the data file in block 136, and providing access to the data file via the unique identifier after the event in block 138. The method for using quality control information obtained from a microelectrode array before an event 160 includes the steps of obtaining quality control information from the microelectrode array after the event in block 162, accessing the quality control information obtained before the event in block 164, and comparing the quality control information obtained before the event with the quality control information obtained after the event in block 166. Method 130 may also include the steps of growing a cell culture on the microelectrode array and assembling a chamber that includes the cell culture grown on the microelectrode array and that can be used to ship and test the cell culture.

A combined method may include the following process steps:

1) grow cell cultures on electrode plates

2) package cell cultures in testing/shipping chamber

3) test spontaneous activity

4) create data file with active electrodes and wave form signals

5) package cell cultures on electrode plates for shipping

6) send cell culture on electrode plates to recipient for experimentation

7) send data file to recipient to facilitate the identification of active electrodes and wave forms

8) recipient inserts cell culture and data file to test station to experimentation

9) test station uses data file as basis for auto-identification of active electrodes and wave forms

10) recipient begins experimentation

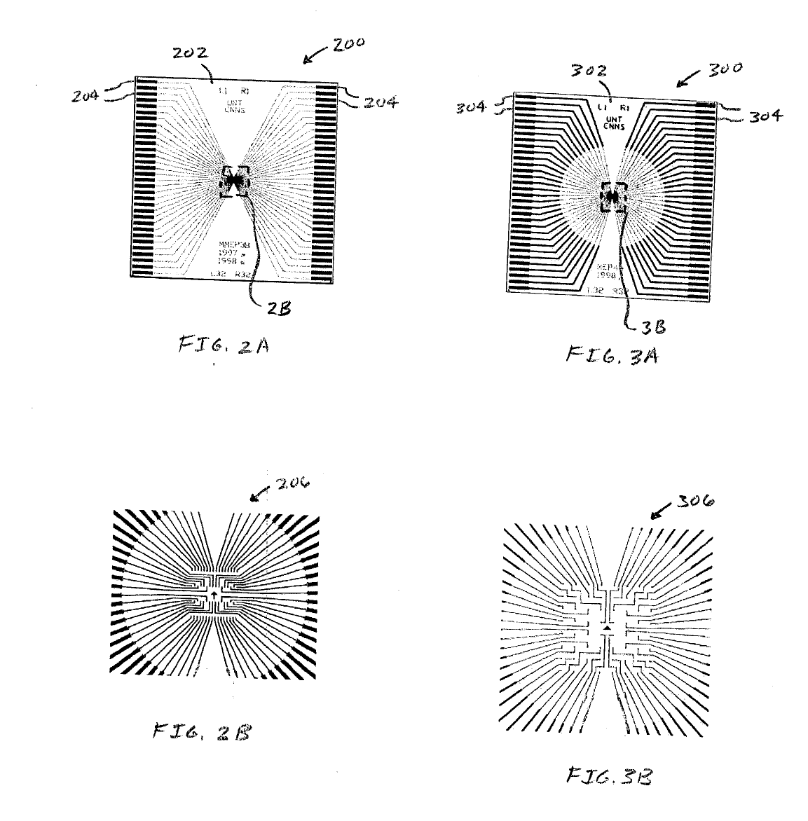

Referring now to FIG. 2, a typical microelectrode arrays (MEA detectors) that can be used in connection with the present invention are illustrated. Microelectrode array is a substrate or carrier plate having a number of electrodes within a recording area (expanded on right) at the center of the substrate (on left). Each electrode is electrically connected to a terminal at the edge of the substrate. During use, the terminals are communicably coupled to the data capture unit 106 (FIG. 1). As more clearly shown in FIG. 2, a 64 conductor MMEP 3B (product of the Center for Network Neuroscience) terminates in a 1.2 mm² recording area (on right) having 8 rows of 8 columns. The electrode spacing is 150 μm between electrodes and 150 μm between rows. The carrier plate (on left) measures 5×5 cm and is 1.1 mm thick.

Referring now to FIGS. 2A, 2B, 3A and 3B, typical microelectrode arrays (MEA detectors) 200 and 300 that can be used in connection with the present invention are illustrated. Microelectrode array 200 is a substrate or carrier plate 202 having a number of electrodes within a recording area 206 (FIG. 2B) at the center of the substrate 202. Each electrode is electrically connected to a terminal 204 at the edge of the substrate 202. During use, the terminals are communicably coupled to the data capture unit 108 (FIG. 1). As more clearly shown in FIG. 2B, a 64 conductor MMEP 3B (product of the Center for Network Neuroscience) terminates in a 0.8 mm² recording area 206 having 4 rows of 16 columns. The electrode spacing is 40 μm between electrodes and 200 μm between rows. The electrode area is roughly 200 μm². The carrier plate 202 measures 5×5 cm and is 1.1 mm thick.

Similarly, microelectrode array 300 is a substrate or carrier plate 302 having a number of electrodes within a recording area 306 (FIG. 3B) at the center of the substrate 302. Each electrode is electrically connected to a terminal 304 at the edge of the substrate 302. During use, the terminals are communicably coupled to the data capture unit 108 (FIG. 1). As more clearly shown in FIG. 3B, a 64 conductor MMEP 4A terminates in a 1.2 mm recording area 306 having a matrix of 8 rows by 8 columns. Electrode spacing is equidistant at 150 μm. Electrode area is roughly 900 μm². The carrier plate 302 measures 5×5 cm and is 1.1 mm thick.

The microelectrode arrays 200 and 300 are fabricated from a base of quartz coated borosilicate glass sputter coated with a layer of indium tin oxide. The indium tin oxide is photo-etched into a micro-circuit pattern and insulated with polysiloxane. The electrode pads are de-insulated with a laser and electroplated with gold. The insultation, which is normally hydrophobic, is rendered hydrophilic by flaming with a butane torch and then coated with poly-D-lysine and laminin to promote cell adhesion.

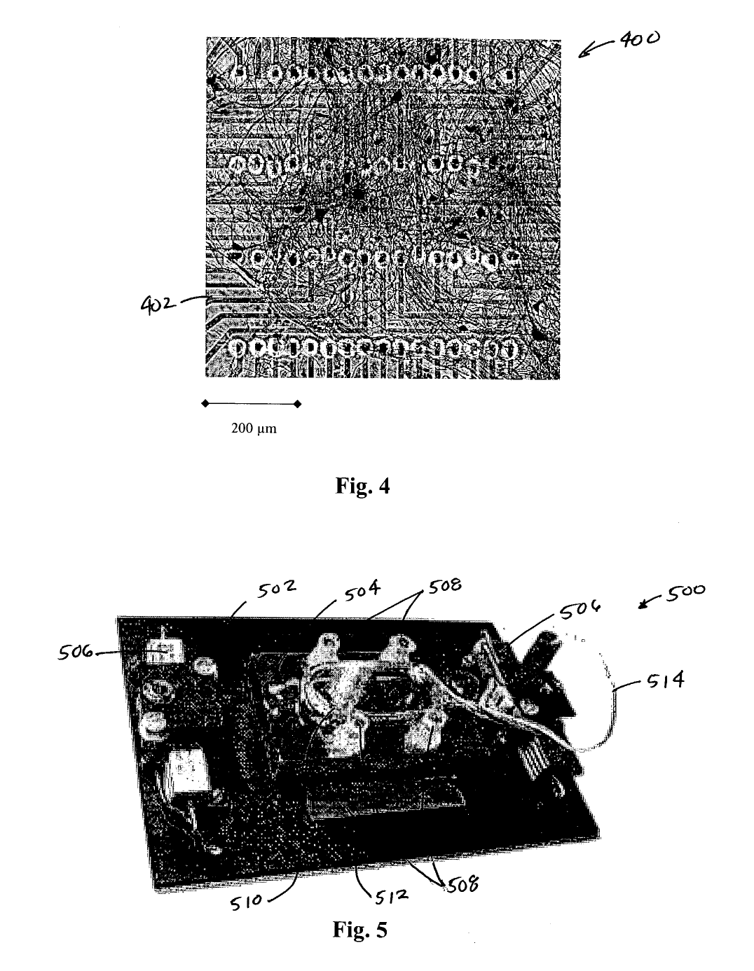

Now referring to FIG. 4, a photograph 400 of neuronal cells grown on a microelectrode array 402 is shown. The microelectrode array 402 is a 64 conductor, MMEP 3B (product of the Center for Network Neuroscience), with a 0.8 mm² recording area having a matrix of 4 rows by 16 columns. Electrode spacing is 40 μm between electrodes and 200 μm between rows. The neuronal cells are grown from frontal cortex cell extracted from a pre-natal mouse. An electrode can record action potentials from the neuronal cells whose cell bodies or axons are in close proximity to the electrode.

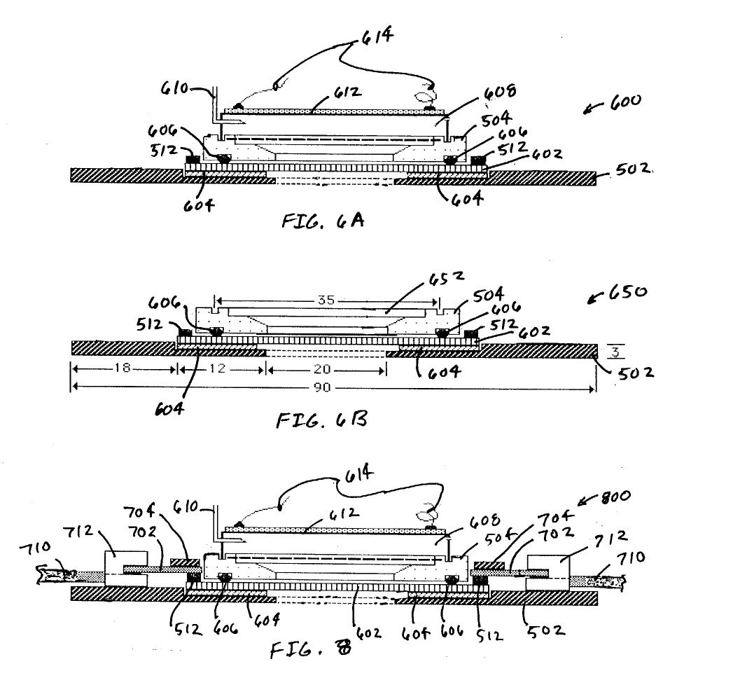

Referring now to FIG. 5, a photograph of a dual network shipping chamber 500 is shown. The shipping chamber 500 has a base plate 502 that supports the microelectrode arrays (not shown) and the stainless steel medium chamber 504 that covers the microelectrode arrays (not shown). Note that the dual network shipping chamber 500 contains two microelectrode arrays (not shown); whereas the single network shipping chamber 600 of FIG. 6 contains one microelectrode array. The shipping chamber 500 can be designed to contain more than two microelectrode arrays. The environment of the dual network shipping chamber 500 can be controlled (e.g., CO₂, temperature, etc.) or monitored (e.g., stress, temperature, etc.) during shipment via an external device. Resisters 506 (optional) on the ends of the base plate 502 are used for heating the chamber 500. Plastic leur-type fittings or ports 508 protrude from the medium chamber 504. These ports 508 are used to perform medium exchanges during testing. A lid or cover 510 is used during testing to maintain a 10% CO₂ atmosphere above the cell culture medium. During shipping, this cover 510 is replaced by a cover which seals the top of the medium chamber 504 to prevent leaks during shipment. A zebra strip 512 or other type of connection interface extends out from the microelectrode array (not shown) to allow a testing apparatus to interface with the microelectrode array (not shown). Thermister wire 514 is used in controlling environment of the medium chamber 504.

Now referring to FIG. 6A, a cross section of a shipping chamber 600 in an open chamber configuration is shown. The shipping chamber 600 includes a microelectrode array 602 disposed on a support bed 604, which is disposed within an inset area of the base plate 502. The support bed 604 cushions the connection between the base plate 502 and the microelectrode array 602. The medium chamber 504 is disposed on top of O-rings 606 that rest on the top of the microelectrode array 602 to cushion the microelectrode array 602 and seal the bottom portion of the environment. A cover 608 is disposed on top of the medium chamber 504 and is equipped with a CO₂ feed 610, a heater plate 612 and thermister wires 614. A zebra strip 512 or other type of connection interface extends out from the microelectrode array 602 to allow a testing apparatus to interface with the microelectrode array 602.

Referring now to FIG. 6B, a cross section of a shipping chamber 650 in a closed chamber configuration is shown. The shipping chamber 650 includes a microelectrode array 602 disposed on a support bed 604, which is disposed within an inset area of the base plate 502. The support bed 604 cushions the connection between the base plate 502 and the microelectrode array 602. The medium chamber 504 is disposed on top of O-rings 606 that rest on the top of the microelectrode array 602 to cushion the microelectrode array 602 and seal the bottom portion of the environment. A cover 652 is disposed on top of the medium chamber 504. A zebra strip 512 or other type of connection interface extends out from the microelectrode array 602 to allow a testing apparatus to interface with the microelectrode array 602.

Now referring to FIG. 7, a photograph of the integration of the shipping chamber with a data capture unit (collectively 700) is shown. The shipping chamber shown is a single network chamber (Center for Network Neuroscience at the University of North Texas) as previously described in reference to FIG. 6A. A cover 608 is disposed on top of the medium chamber 504 and is equipped with a CO₂ feed 610, a heater plate 612 and thermister wires 614 to control the environment of the cell culture during testing. Printed circuit boards extensions 702 extend out over the edges of the shipping chamber to be electrically connected to circuit board or zebra strip that is connected to the microelectrode array. The electrical connections are maintained using pressure plates 704 and thumb screws 706. The pre-amplifiers 708 of the data capture unit (Plexon) are mounted on the printed circuit boards 710, which are connected to the printed circuit board extensions 702 with connectors 712.

Now referring to FIG. 8, a cross section of a shipping chamber (open chamber configuration) connected to a data capture unit (collectively 800) is shown. The shipping chamber includes a microelectrode array 602 disposed on a support bed 602, which is disposed within an inset area of the base plate 502. The support bed 602 cushions the connection between the base plate 502 and the microelectrode array 602. The medium chamber 504 is disposed on top of O-rings 606 that rest on the top of the microelectrode array 602 to cushion the microelectrode array 602 and seal the bottom portion of the environment. A cover 608 is disposed on top of the medium chamber 504 and is equipped with a CO₂ feed 610, a heater plate 612 and thermister wires 614. A zebra strip 512 or other type of connection interface extends out from the microelectrode array 602 to allow a testing apparatus to interface with the microelectrode array 602. Printed circuit boards extensions 702 extend out over the edges of the shipping chamber to be electrically connected to circuit board or zebra strip 512 that is connected to the microelectrode array 602. The electrical connections are maintained using pressure plates 704 and thumb screws (not shown). The pre-amplifiers of the data capture unit (Plexon) are mounted on the printed circuit boards 710, which are connected to the printed circuit board extensions 702 with connectors 712.

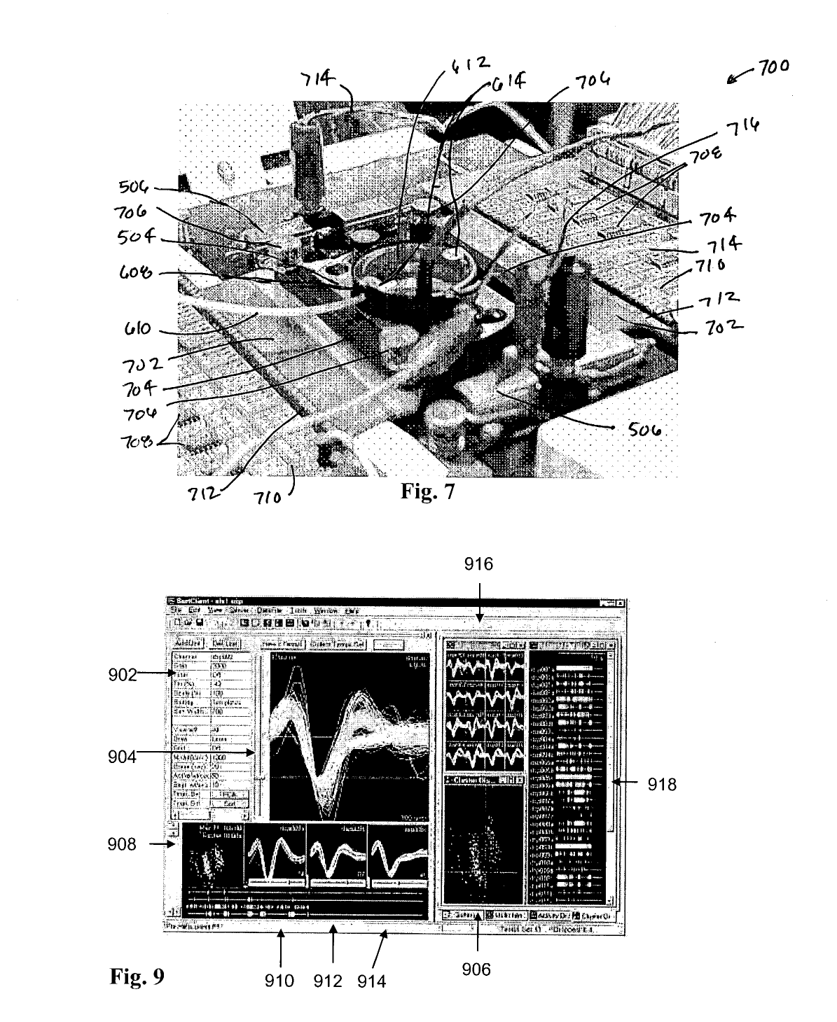

Referring now to FIG. 9, a screen image 900 from a standard extracellular recording software program (Sort Client software from Plexon) is shown. The setting table 902 lists the settings of the channel being viewed. A graph of all of the data collected from a single channel (or electrode) is shown by 904 and 906. Different colors of the waveforms represent different waveform signals that fit different waveform templates and likely originate from different neurons. The two dimensional representation 908 of all of the waveforms recorded from the channel being viewed. Three smaller graphs 910, 912 and 914 include only grouped waveforms representing signals from individual waveforms. A selection of waveforms 916 from individual neurons that can be recorded in the data file as individual units. The rastor plot 918 shows the timing of the action potentials recorded on each of the selected units. Each vertical marker on the horizontal timeline represents one action potential from the selected unit. Multiple companies sell waveform data collection and analysis software.

The data included in the data file may include, but is not limited to:

Number of SIG, DSP, OUT and BNC channels

SIG/DSP assignments

OUT assignments

BNC assignments

Per-channel gain and filter settings

Number of points per waveform

Number of prethreshold points

Maximum waveforms/second to display for main (monitored) channel

Maximum waveforms/second to display for other channels

NIDAQ (slow channel) digitizing rate

Template adjustment amount (adaptive waveform tracking)

Template adjustment threshold

Gain multiplier

External event names, channels

For each slow channel:

NIDAQ gain

preamp gain

For each recording channel:

Name

assigned SIG

SIG name

Gain

display scale

filter on/off

sorting method

threshold

number of sorted units

sorting parameters:

rectangles or waveform templates

waveform width for sorting

maximum waveforms/second

Hardware configuration

Interface (MXI, HLK2, serial) configuration

MAP digitizing rate

MAP polling rate

MAP DSP program file

NIDAQ configuration

Memory mapped file configuration

Thread properties

Examples of three (3) data files in accordance with the present invention are included in a computer program listing appendix “A” on a compact disc. The material of the disc is incorporated herein by reference. The three (3) data files are a PLAC Plate Client data file, a FEC front end client data file and a sorting client data file identified as “PLAC.txt”, “FEC.txt” and “Sort.txt” respectively.

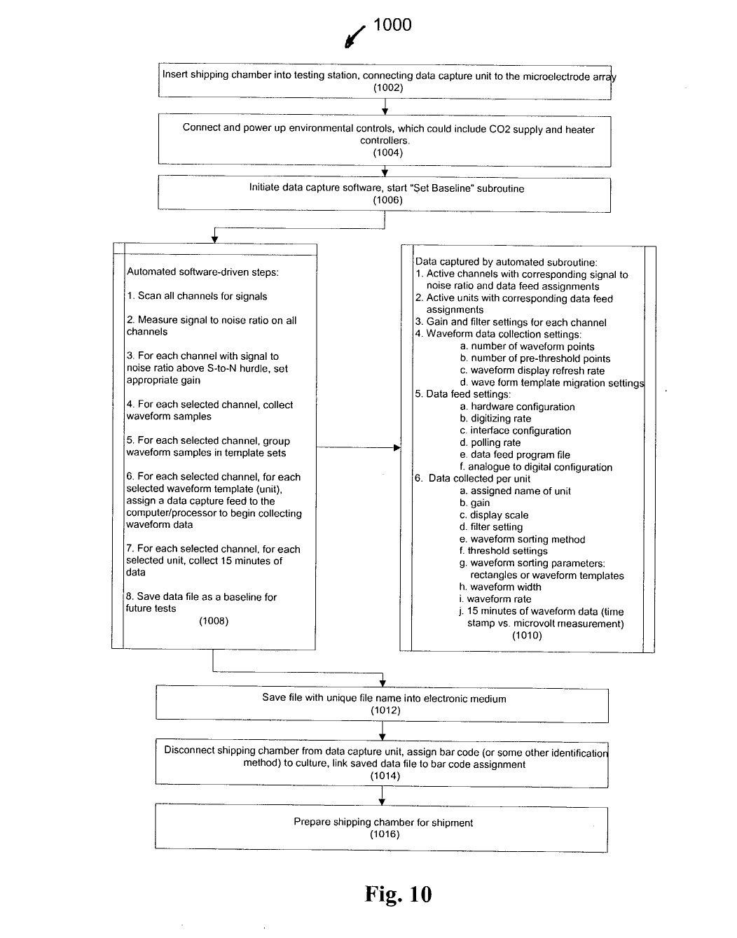

Referring now to FIG. 10, a flow chart describes the pre-shipment data collection process 1000. Those skilled in extracellular recording can grow neuronal cultures on microelectrode arrays and assemble the shipping chamber. Once the chamber is assembled, the pre-amplifiers from a data capture unit are attached to the shipping chamber in block 1002. The environmental controls are then attached and the chamber is brought up the testing environment, which could be 37° C. and 10% CO₂ in block 1004. Osmolarity of the cell culture medium is tested and adjusted if required. The “Set Baseline” subroutine is initiated to begin the pre-shipment data collection in block 1006. While this subroutine could be an external program, the integration of this subroutine into a standard data capture software program is useful and desirable. As shown in block 1008, the subroutine scans all channels (electrodes) for signal activity (step 1), measures the signal-to-noise ratio on all the channels (step 2), and automatically assigns a gain setting to each channel as the subroutine finds channels which meet certain signal-to-noise criteria, which are set by the user (step 3). For each active channel identified by the subroutine, the subroutine assigns collects a sample, which could be at least one hundred, of waveforms (step 4). Using a grouping algorithm, the subroutine is able to separate multiple signals from one channel, recorded from different neurons. A unit template is assigned to each waveform grouping (step 5). After all active channels have been separated into active units and assigned a data capture feed to the computer or processor to collect the waveform data (step 6), the subroutine records data for a period of time, which could be 15 minutes (step 7). This data is saved into a file that can be used as a baseline for future tests (step 8). The file is saved into an electronic medium with a unique name or identifier that is linked to a bar code (or some other tracking method) on the shipping chamber in block 1012. The chamber is then prepared for shipping in block 1016.

The data recorded by the subroutine in block 1008 and linked to the cell cultures provides the baseline to which the neuroactivity of the cell culture can be compared after a period of time. The settings recorded in the data file in block 1012 are used during the next test of the cell culture to enable a fair comparison of the waveforms collected. As shown in block 1010, the data captured by the subroutine may include: (1) active channels with corresponding signal-to-noise ratio and data feed assignments; (2) active units with corresponding data feed assignments; (3) gain and filter settings for each channel; (4) waveform data collection settings (number of waveform points, number of pre-threshold points, waveform display refresh rate, waveform template migration settings); (5) data feed settings (hardware configuration, digitizing rate, interface configuration, polling rate, data feed program file, analog to digital configuration); and (6) data collected per unit (assigned name of unit, gain, display scale, filter setting, waveform sorting method, threshold settings, waveform sorting parameters, waveform width, waveform rate, waveform data collected over the time period). The recorded waveforms are compared with future tests of the culture to measure changes in the neuroactivity of the culture, which provides an assessment of the quality of the culture over time and before and after specific events.

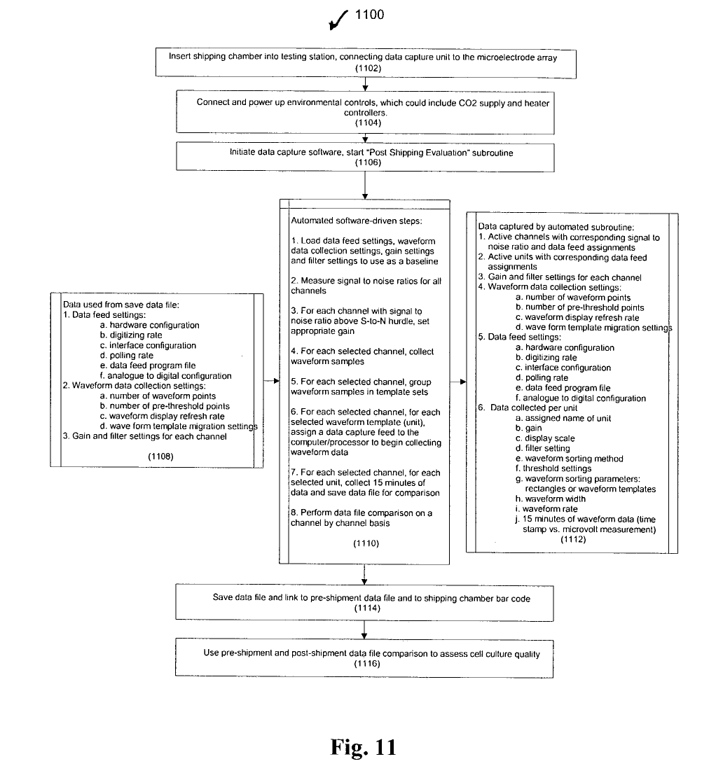

Now referring to FIG. 11, a flow chart describes the post-shipment testing process 1100. Those skilled in extracellular recording can attached the pre-amplifiers from a data capture unit to the shipping chamber in,block 1102. The environmental controls are then attached and the chamber is brought up the testing environment, which could be 37° C. and 10% CO₂ in block 1104. Osmolarity of the cell culture medium is tested and adjusted if required. The “Post Shipment Evaluation” subroutine is initiated to begin the post-shipment data collection in block 1106. While this subroutine could be an external program, the integration of this subroutine into a standard data capture software program is useful and desirable. As shown in block 1110, the subroutine loads the settings (data feed settings, waveform data setting, collection settings, gain settings and filter settings) from the pre-shipment data file 1108 and uses those settings as a starting point (baseline) for a scan of all channels (electrodes) for signal activity (step 1). The signal-to-noise ratios for all channels are then measured (step 2). The subroutine 1010 automatically assigns a gain setting to each channel for those channels that meet certain signal-to-noise criteria (step 3), which are referenced from the pre-shipment data file 1108. For each active channel identified by the subroutine, the subroutine assigns collects a sample, which could be at least one hundred, of waveforms (step 4). Using a grouping algorithm, the subroutine 906 is able to separate multiple signals from one channel, recorded from different neurons. A unit template is assigned to each waveform grouping (step 5). After all active channels have been separated into active units and assigned a data capture feed to the computer or processor to collect the waveform data (step 6), the subroutine records data for a period of time, which could be 15 minutes (step 7). A data file comparison on a channel-by-channel basis can then be performed (step 8). This data is saved into a file 1114 that is linked to pre-shipment data file 1014 (FIG. 9) and the bar code (or some other tracking method) assigned during pre-shipment testing. A comparison is performed by the subroutine to assess the changes between pre-shipment activity and post-shipment activity in block 1116.

As shown in block 1112, the data recorded by the subroutine in block 1110 may include: (1) active channels with corresponding signal-to-noise ratio and data feed assignments; (2) active units with corresponding data feed assignments; (3) gain and filter settings for each channel; (4) waveform data collection settings (number of waveform points, number of pre-threshold points, waveform display refresh rate, waveform template migration settings); (5) data feed settings (hardware configuration, digitizing rate, interface configuration, polling rate, data feed program file, analog to digital configuration); and (6) data collected per unit (assigned name of unit, gain, display scale, filter setting, waveform sorting method, threshold settings, waveform sorting parameters, waveform width, waveform rate, waveform data collected over the time period). The recorded waveforms are compared with future tests of the culture to measure changes in the neuroactivity of the culture, which provides an assessment of the quality of the culture over time and before and after specific events.

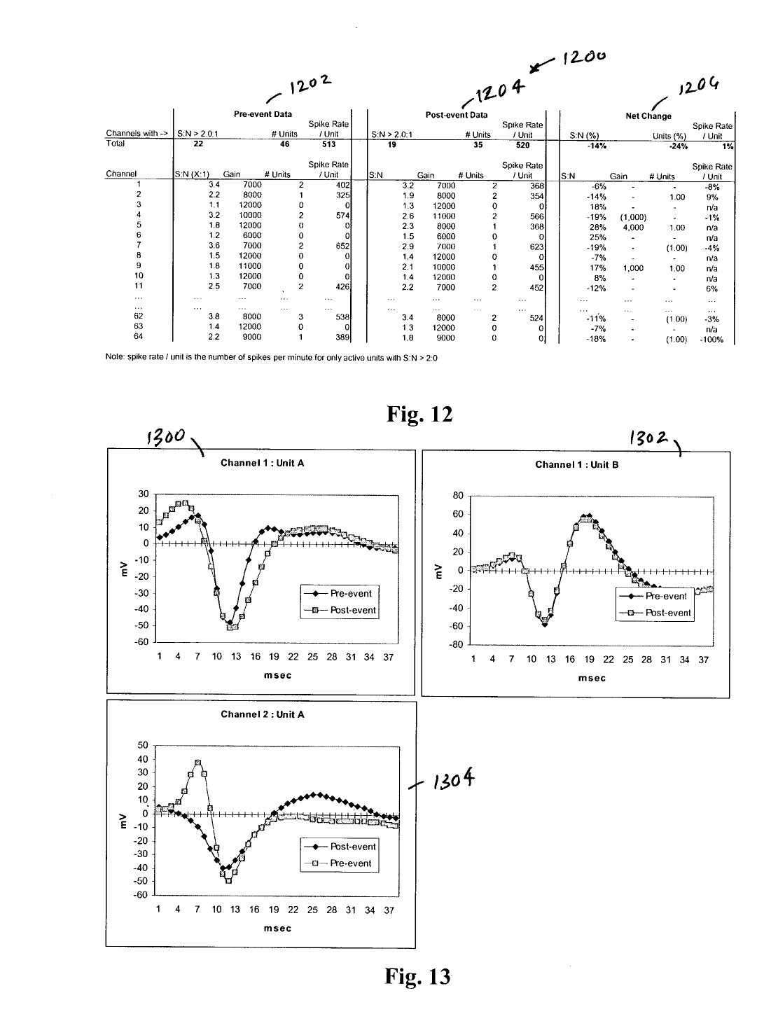

Referring now to FIG. 12, a sample pre-event and post-event channel comparison (collectively 1200) is shown. An event, such as shipping, causes stress to a cell culture. Sample data collected using the present invention before an event (Pre-event Data 1202) after an event (Post-event Data 1204) shows the loss of 3 channels out (14%) of 22 channels with a signal to noise ratio over 2:1 (Net Change 1206), which is the criteria for a channel to be considered active and counted by the Center for Network Neuroscience (Table 1). The over all unit loss during the event was 11 units (24%). The spike rate per active unit changed only slightly during the event. FIG. 12 displays an overall comparison as well as a channel by channel comparison. The channel by channel comparison can be used in conjunction with electrode-cell coupling inspections, cell health inspections and other inspections to determine the causes of the changes induced by the event.

Now referring to FIG. 13, a sample pre-event and post-event unit waveform comparison 1300, 1302 and 1304 is shown. Waveforms recorded during the pre-event quality characterization are compared to waveforms recorded during the post-event characterization. The matching waveforms as shown in the graph for Channel 1: Unit B 1302 signify that the recordings are likely from the same neuron. Non-matching signals as shown in the graph for Channel 2: Unit A 1304 signify that the recordings may not be from the same neuron. If, as in the graph from Channel 2: Unit A 1304, there has been a shift in the recorded neuron, then there could have been movement of the axons or cell bodies above the electrode corresponding to that channel. The purpose of recording such data is to show trends that could be correlated to other, more specific data on the cell cultures.

While this invention has been described in reference to illustrative embodiments, this description is not intended to be construed in a limiting sense. Various modifications and combinations of the illustrative embodiments, as well as other embodiments of the invention, will be apparent to persons skilled in the art upon reference to the description. It is therefore intended that the appended claims encompass any such modifications or embodiments.

What is claimed is:

1. A system for providing quality control information pertaining to a cell culture comprising:
a chamber containing the cell culture; and
a unique identifier disposed on the chamber corresponding to a data file containing the quality control information pertaining to the cell culture before an event.

2. The system as recited in claim 1, wherein the event is shipping the chamber.

3. The system as recited in claim 1, wherein the event is adding a compound to the cell culture.

4. The system as recited in claim 1, wherein the event is performing a procedure on the cell culture.

5. The system as recited in claim 1, wherein the quality control information comprises baseline testing information.

6. The system as recited in claim 1, wherein the quality control information comprises:
an identification of each active electrode on which the cell culture are present; and
one or more waveform templates for each active electrode.

7. The system as recited in claim 1, wherein the unique identifier comprises a memory device containing the data file.

8. The system as recited in claim 7, wherein the memory device a flash memory.

9. The system as recited in claim 7, wherein the memory device is a USB key.

10. The system as recited in claim 1, wherein the unique identifier corresponds to a computer readable medium where the data file is stored.

11. The system as recited in claim 10, wherein the computer readable medium is a floppy disk.

12. The system as recited in claim 10, wherein the computer readable medium is a smart card.

13. The system as recited in claim 10, wherein the computer readable medium is a CD-ROM.

14. The system as recited in claim 1, wherein the unique identifier is a bar code.

15. The system as recited in claim 1, wherein the unique identifier is a series of alphanumeric characters.

16. The system as recited in claim 1, wherein the data file is accessible via the Internet.

17. The system as recited in claim 1, wherein the data file is delivered via e-mail.

18. The system as recited in claim 1, wherein the chamber is a shipping chamber.

19. The system as recited in claim 1, wherein the chamber is a testing chamber.

20. The system as recited in claim 1, wherein the cell culture comprises neuronal cells.

21. The system as recited in claim 20, wherein the neuronal cells comprise an embryonic stem cell from a knock-out, knock-in, over-expressing transgenic, under-expressing-transgenic, a conditional knockout, a mutant and the like.

22. The system as recited in claim 20, wherein the neuronal cells are from an animal knock-out, knock-in, over-expressing transgenic, under-expressing-transgenic, a conditional knockout, a mutant and the like.

23. The system as recited in claim 20, wherein the neuronal cells are selected from the frontal cortex, the auditory cortex, the visual cortex, the hippocampus or the spinal cord.

24. A method for providing quality control information obtained from a microelectrode array before an event comprising the steps of:
obtaining quality control information from the microelectrode array before the event;
storing the quality control information in a data file;
associating a unique identifier with the microelectrode array that corresponds to the data file; and
providing access to the data file via the unique identifier after the event.

25. The method as recited in claim 24, further comprising the steps of:
growing a cell culture on the microelectrode array; and
assembling a chamber that includes the cell culture grown on the microelectrode array and that can be used to ship and test the cell culture.

26. A method for using quality control information obtained from a microelectrode array before an event comprising the steps of:
obtaining quality control information from the microelectrode array after the event;
accessing the quality control information obtained before the event; and
comparing the quality control information obtained before the event with the quality control information obtained after the event.

27. A method for using quality control information obtained from a microelectrode array comprising the steps of:
obtaining quality control information from the microelectrode array before an event;
storing the quality control information obtained before the event in a data file;
associating a unique identifier with the microelectrode array that corresponds to the data file;
obtaining quality control information from the microelectrode array after the event;
accessing the quality control information obtained before the event via the unique identifier; and
comparing the quality control information obtained before the event with the quality control information obtained after the event.

28. A method for providing an automated quality characterization of a cell culture grown on a microelectrode array for the purpose of measuring the effects on the cell culture before and after an event comprising the steps of:
growing the cell culture from a wild-type or genetically modified animal on the microelectrode array;
assembling a chamber that includes the cell culture grown on the microelectrode array and that can be used to ship and test the cell culture;
measuring an electrical activity of the cell culture before an event in order to characterize a quality of the cell culture;
storing quality control information pertaining to the cell culture, which includes both software settings and the measured electrical activity of the cell culture, on a computer readable medium for the purposes of reference at the time of subsequent testing of the cell culture;
measuring the electrical activity of the cell culture after the event; and
comparing the quality characterization measurements from the cell culture before and after the event to assess the effects of the event on the cell culture and the usability of the cell culture for future experiments.

29. The method as recited in claim 28, wherein the cell culture comprises an embryonic stem cell from a knock-out, knock-in, over-expressing transgenic, under-expressing-transgenic, a conditional knockout, a mutant and the like.

30. The method as recited in claim 28, wherein the cell culture comprises cells selected from the frontal cortex, the auditory cortex, the visual cortex, the hippocampus or the spinal cord.
